(19) United States
(12) Patent Application Publication
(10) Pub. No.: US 2015/0177229 A1
(43) Pub. Date: Jun. 25, 2015

(54) Cell Mobility Characteristics Sensing Apparatus and Operating Method Thereof

(71) Applicants: Long Hsu, Hsinchu City (TW); Crystalvue Medical Corporation, Taoyuan (TW)

(72) Inventors: Long HSU, Hsinchu City (TW); William WANG, Taoyuan City (TW); Sheng-Hsiang LI, Taoyuan (TW); Chung-Hao LU, Taoyuan (TW); Cheng-Hsien LIU, Hsinchu City (TW); Yuh-Shyong YANG, Hsinchu City (TW); Hwan-You CHANG, Hsinchu City (TW); Sung-Yang WEI, Taoyuan (TW); Chung-Cheng CHOU, Taoyuan County (TW)

(21) Appl. No.: 14/583,003

(22) Filed: Dec. 24, 2014

(30) Foreign Application Priority Data
Dec. 24, 2013 (TW) 102147985

(51) Int. Cl. G01N 33/50; G01N 21/51

(52) U.S. Cl. CPC G01N 33/5091; G01N 21/51; G01N 2201/06113; G01N 2800/367

(57) ABSTRACT

A cell mobility characteristics sensing apparatus including a laser light source, a light sensor, an analyzing chip, and a display is disclosed. The laser light source emits laser beams to a cell sample. The light sensor senses scattered laser beams formed by the cell sample scattering the laser beams at a plurality of time points to obtain a plurality of laser scattering patterns corresponding to the plurality of time points respectively. The analyzing chip obtains a laser scattering pattern fluctuations information of the plurality of laser scattering patterns varied with the plurality of time points to estimate the mobility characteristics of the cells in the cell sample. The display shows the mobility characteristics of the cells in the cell sample estimated by the analyzing chip.

### BACKGROUND OF THE INVENTION

1. Field of the Invention

This invention relates to cell mobility characteristics, especially to a cell mobility characteristics sensing apparatus and an operating method thereof.

2. Description of the Prior Art

The mobility of sperms is an important indicator to determine whether the patient's sperms are normal, and its value represents the percentage of sperms having a moving speed larger than 25 um/sec in a sperm sample. For example, if the mobility of sperms is 50%, it represents that the moving speed of 50% of the sperms in the sperm sample is larger than 25 um/sec. Therefore, the mobility of sperms can be a quantifiable indicator to indicate that the quality of the patient's sperms is good or poor. This is also currently used by many reproductive medicine laboratories to determine the quality of the patient's sperms. If the quality of the patient's sperms is not good enough, they can provide early artificial insemination treatment to the patent.

In general, hospitals usually determine the mobility of sperms by microscope observations or the data provided by the computer assisted sperm analyzer (CASA). However, the microscope observations fail to quantify the mobility of sperms. Although the CASA has many functions, the CASA is expensive and has huge volume, and the operation of the CASA is complicated. That is to say, it is not suitable for ordinary person to use the microscope and CASA to sense the mobility of sperms at home.

Therefore, the invention provides to a cell mobility characteristics sensing apparatus and an operating method thereof solve the above-mentioned problems.

### SUMMARY OF THE INVENTION

An embodiment of the invention is a cell mobility characteristics sensing apparatus.

In this embodiment, the cell mobility characteristics sensing apparatus is used for sensing mobility characteristics of a plurality of cells in a cell sample. The cell mobility characteristics sensing apparatus includes a laser light source, a light sensor, an analyzing chip, and a display. The laser light source is used for emitting a laser beam to the cell sample. The light sensor is used for sensing scattered laser beams formed by the cell sample scattering the laser beam at a plurality of time points to obtain a plurality of laser scattering patterns corresponding to the plurality of time points respectively. The analyzing chip is coupled to the light sensor and used for obtaining a laser scattering pattern fluctuations information of the plurality of laser scattering patterns varied with the plurality of time points to estimate the mobility characteristics of the plurality of cells in the cell sample. The display is coupled to the analyzing chip and used for showing the mobility characteristics of the plurality of cells in the cell sample estimated by the analyzing chip.

In an embodiment, the mobility characteristics are mobility, quantity, moving velocity distribution, or linearity of moving path.

In an embodiment, the laser scattering pattern fluctuations information includes upper and lower volatility of a plurality of voltages corresponding to the plurality of laser scattering patterns varied with the plurality of time points.

In an embodiment, the more the upper and lower volatility of the plurality of voltages varied with the plurality of time points, the higher the mobility of the plurality of cells in the cell sample estimated by the analyzing chip.

In an embodiment, the light sensor obtains the plurality of voltages corresponding to the plurality of laser scattering patterns through a photoresistor or a photodiode.

Another embodiment of the invention is a method of operating a cell mobility characteristics sensing apparatus. In this embodiment, the method is used for sensing mobility characteristics of a plurality of cells in a cell sample. The cell mobility characteristics sensing apparatus includes a laser light source, a light sensor, an analyzing chip, and a display. The method includes steps of: (a) the laser light source emitting a laser beam to the cell sample; (b) the light sensor sensing scattered laser beams formed by the cell sample scattering the laser beam at a plurality of time points to obtain a plurality of laser scattering patterns corresponding to the plurality of time points respectively; (c) the analyzing chip obtaining a laser scattering pattern fluctuations information of the plurality of laser scattering patterns varied with the plurality of time points to estimate the mobility characteristics of the plurality of cells in the cell sample; and (d) the display showing the mobility characteristics of the plurality of cells in the cell sample estimated by the analyzing chip.

Compared to the prior art, the cell mobility characteristics sensing apparatus and the operating method thereof have advantages of:

(1) simple structure and low cost;

(2) capable of calculating many cell parameters such as mobility, quantity, moving velocity distribution, and linearity of moving path and being widely applied to sense different kinds of cells;

(3) easy and fast operation for the patient to do the initial detection of the cell mobility at home; if the sensing result is abnormal, the patient can go to hospital to do more detailed examinations.

The advantage and spirit of the invention may be understood by the following detailed descriptions together with the appended drawings.

### BRIEF DESCRIPTION OF THE APPENDED DRAWINGS

FIG. 1 illustrates a schematic diagram of the cell mobility characteristics sensing apparatus in an embodiment of the invention.

FIG. 2 illustrates a schematic diagram of the cell mobility characteristics sensing apparatus in another embodiment of the invention.

FIG. 3A and FIG. 3B illustrates schematic diagrams of the first cell sample having fewer cells and the second cell sample having more cells respectively.

FIG. 4A and FIG. 4B illustrates diagrams of the voltages of the first cell sample and the second cell sample varied with time respectively.

FIG. 5 illustrates diagrams of the voltages of the different sperm samples SS1˜SS3 varied with time respectively.

FIG. 6 illustrates a flowchart of the cell mobility characteristics sensing apparatus operating method in another embodiment of the invention.

### DETAILED DESCRIPTION OF THE INVENTION

In general, the mobility of sperms is an important indicator to determine the degree of difficulty of a successful pregnancy; therefore, if there is a sperm mobility sensing apparatus which can be easily and fast operated, the patient can use it to do the initial sensing of the sperm mobility at home. If the sensing result is abnormal, the patient can go to hospital to do more detailed examinations. It helps the patient to receive early artificial insemination treatment.

It is possible to use an optical method to sense the mobility of sperms. Assuming a bunch of laser lights as a light source, it can be found that when the sperms on a glass slide is irradiated under the laser beam, the laser beam will be scattered by the sperms and the scattering patterns seem to correspond to the mobility of sperms. For example, if the sperms have higher mobility, the scattering patterns scattered by the sperms will flash with time; if the sperms have poor mobility, the scattering patterns scattered by the sperms will maintain unchanged. Thus, the characteristic speed and mobility of the sperms can be reflected by the degree of the flashing of the scattering patterns mentioned above.

Accordingly, the present invention is intended to design a cell mobility characteristics sensing apparatus including a light source, a low-level light sensing chip, and a microprocessor to quantify the sperm mobility according to the changes of the flashing of the laser scattering patterns scattered by the sperms at different time points and try to get more information about the movement of the sperms from the scattered lights. As long as the degree of the flashing of the laser scattering patterns varied with time is quantified and compared with the sperm mobility data obtained by the CASA, the information about sperm mobility and sperm density can be calculated according to the laser scattering pattern fluctuations information.

The invention is done cooperated with the reproductive and endocrine laboratory of the Mackay memorial hospital to use an advanced computer sperm analyzing system to determine the mobility of the sperms (the percentage of sperms having a moving speed larger than 25 um/sec in the sperm sample) through a light tracing method and to calculate sperm parameters such as sperm quantity, sperm moving velocity distribution, or linearity of sperm moving path at the same time. It is a great tool to analyze the movement of the sperms.

A preferred embodiment of the invention is a cell mobility characteristics sensing apparatus. In this embodiment, the cell mobility characteristics sensing apparatus is used for sensing mobility characteristics of a plurality of cells in a cell sample, such as parameters of cell mobility, cell quantity, cell moving velocity distribution, and linearity of cell moving path, but not limited to this.

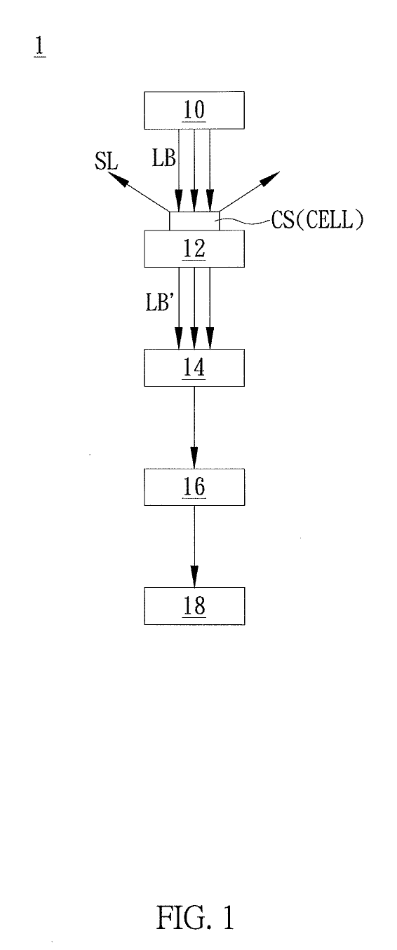

Please refer to FIG. 1. FIG. 1 illustrates a schematic diagram of the cell mobility characteristics sensing apparatus in this embodiment. As shown in FIG. 1, the cell mobility characteristics sensing apparatus 1 includes a laser light source 10, a sample platform 12, a light sensor 14, an analyzing chip 16, and a display 18. Wherein, the analyzing chip 16 is coupled to the light sensor 14; the display 18 is coupled to the analyzing chip 16.

The sample platform 12 is used to dispose the cell sample CS. The cell sample CS includes a plurality of cells CELL. In practical applications, the cells CELL in the cell sample CS can be sperms, flagellated parasites, flagellated algae, or swimming bacteria, but not limited to this.

The laser light source 10 is used to emit a laser beam LB to the cell sample CS disposed on the sample platform 12. The light sensor 14 is used to sense scattered laser beams SL formed by the cell sample CS scattering the laser beam LB and the non-scattered laser beam LB′ at a plurality of time points (e.g., the first time t1˜the fourth time t4) to obtain a plurality of laser scattering patterns corresponding to the plurality of time points t1˜t4 respectively. It should be noticed that the light sensor 14 can have a plurality of light sensing units depending on practical needs. The light sensor 14 can include not only the light sensing units disposed under the cell sample CS, but also the light sensing units disposed at the two sides of the cell sample CS or at the upper left and the upper right of the cell sample CS to sense the scattered laser beams SL scattered by the cell sample CS.

The analyzing chip 16 is used to obtain a laser scattering pattern fluctuations information of the plurality of laser scattering patterns varied with the plurality of time points t1˜t4 to estimate the mobility characteristics of the plurality of cells CELL in the cell sample CS, such as parameters of cell mobility, cell quantity, cell moving velocity distribution, and linearity of cell moving path. The display 18 is used for showing the mobility characteristics of the plurality of cells CELL in the cell sample CS estimated by the analyzing chip 16.

In practical applications, the light sensor 14 can use a photoresistor or a photodiode to obtain a plurality of voltages corresponding to the plurality of laser scattering patterns, but not limited to this. The laser scattering pattern fluctuations information obtained by the analyzing chip 16 can include upper and lower volatility of a plurality of voltages corresponding to the plurality of laser scattering patterns varied with the plurality of time points t1˜t4.

If the upper and lower volatility of the plurality of voltages varied with the plurality of time points t1˜14 is larger, it means that there are larger changes in the plurality of laser scattering patterns obtained at different time points t1˜14; that is to say, the moving of the cells CELL in the cell sample CS is more obvious. Therefore, the analyzing chip 16 will estimate that the cells CELL in the cell sample CS have higher mobility.

On the contrary, if the upper and lower volatility of the plurality of voltages varied with the plurality of time points t1˜t4 is smaller, it means that there are smaller changes in the plurality of laser scattering patterns obtained at different time points t1˜t4; that is to say, the moving of the cells CELL in the cell sample CS is not obvious. Therefore, the analyzing chip 16 will estimate that the cells CELL in the cell sample CS have lower mobility.

In addition, the analyzing chip 16 can also determine the relative quantity of the cells CELL in the cell sample CS according to the average voltage of the voltages obtained at different times t1˜t4 by the light sensor 14. For example, if the average voltage obtained by the light sensor 14 is higher, it means that the relative quantity of the cells CELL in the cell sample CS disposed on the sample platform 12 is smaller, so that only a small part of the laser beam LB is scattered by the cells CELL and the light sensor 14 can receive more laser beam LB; therefore, the voltages obtained by the light sensor 14 through the photoresistor or the photodiode will become higher.

On the contrary, if the average voltage obtained by the light sensor 14 is lower, it means that the relative quantity of the cells CELL in the cell sample CS disposed on the sample platform 12 is larger, so that a large part of the laser beam LB is scattered by the cells CELL and the light sensor 14 can receive less laser beam LB; therefore, the voltages obtained by the light sensor 14 through the photoresistor or the photodiode will become lower.

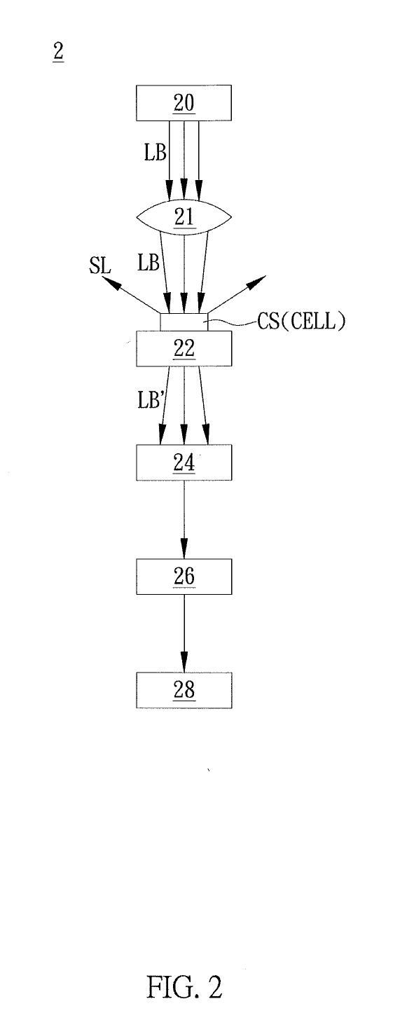

Please refer to FIG. 2. FIG. 2 illustrates a schematic diagram of the cell mobility characteristics sensing apparatus in another embodiment. As shown in FIG. 2, the cell mobility characteristics sensing apparatus 2 includes a laser light source 20, a lens 21, a sample platform 22, a light sensor 24, an analyzing chip 26, and a display 28. Wherein, the analyzing chip 26 is coupled to the light sensor 24; the display 28 is coupled to the analyzing chip 26.

The sample platform 22 is used to dispose the cell sample CS. The cell sample CS includes a plurality of cells CELL. In practical applications, the cells CELL in the cell sample CS can be sperms, flagellated parasites, flagellated algae, or swimming bacteria, but not limited to this.

The laser light source 20 is used to emit a laser beam LB and the laser beam LB is focused by the lens 21 to the cell sample CS disposed on the sample platform 22. The light sensor 24 is used to sense scattered laser beams SL formed by the cell sample CS scattering the focused laser beam LB and the non-scattered laser beam LB′ at a plurality of time points (e.g., the first time t1˜the fourth time t4) to obtain a plurality of laser scattering patterns corresponding to the plurality of time points t1˜t4 respectively.

The analyzing chip 26 is used to obtain a laser scattering pattern fluctuations information of the plurality of laser scattering patterns varied with the plurality of time points t1˜t4 to estimate the mobility characteristics of the plurality of cells CELL in the cell sample CS, such as parameters of cell mobility, cell quantity, cell moving velocity distribution, and linearity of cell moving path. The display 28 is used for showing the mobility characteristics of the plurality of cells CELL in the cell sample CS estimated by the analyzing chip 26.

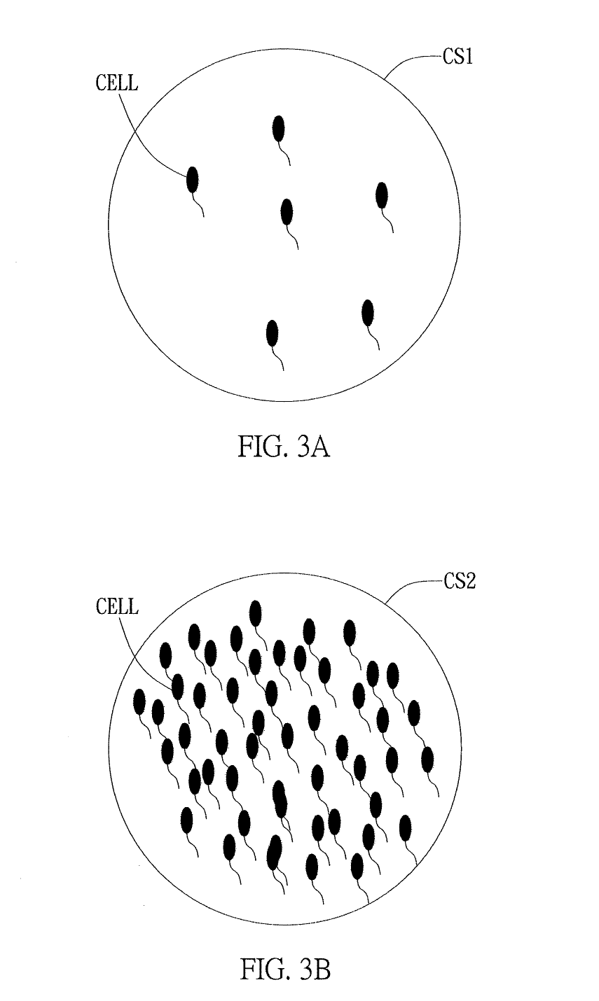

Please refer to FIG. 3A and FIG. 3B. FIG. 3A and FIG. 3B illustrates schematic diagrams of the first cell sample having fewer cells and the second cell sample having more cells respectively. As shown in FIG. 3A and FIG. 3B, if the first cell sample CS1 and the second cell sample CS2 include the same kind of cells CELL, the first cell sample CS1 has fewer cells CELL and the second cell sample CS2 has more cells CELL; that is to say, the cell density of the first cell sample CS1 is smaller than the cell density of second cell sample CS2.

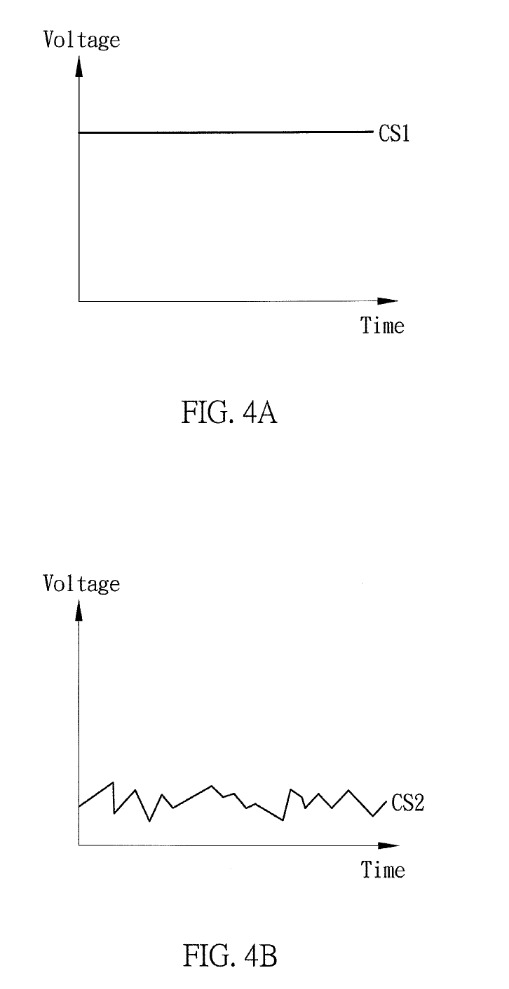

Then, the cell mobility characteristics sensing apparatus of the invention performs the above-mentioned sensing and analyzing operations on the first cell sample CS1 and the second cell sample CS2 respectively to obtain a plurality of voltages at different time points. Please refer to FIG. 4A and FIG. 4B. FIG. 4A and FIG. 4B illustrates diagrams of the voltages of the first cell sample CS1 and the second cell sample CS2 varied with time respectively. As shown in FIG. 4A and FIG. 4B, the voltage of the first cell sample CS1 having fewer cells CELL in FIG. 4A is obviously higher than the voltage of the second cell sample CS2 having more cells CELL in FIG. 4B.

In addition, since the cells CELL of the second cell sample CS2 are far more than the cells CELL of the first cell sample CS1, it can be said that the second cell sample CS2 has more cells CELL having stronger mobility than the first cell sample CS1 does. Therefore, the upper and lower volatility of the plurality of voltages varied with the plurality of time points corresponding to the second cell sample CS2 in FIG. 4B is obviously larger than the upper and lower volatility of the plurality of voltages varied with the plurality of time points corresponding to the first cell sample CS1 in FIG. 4A.

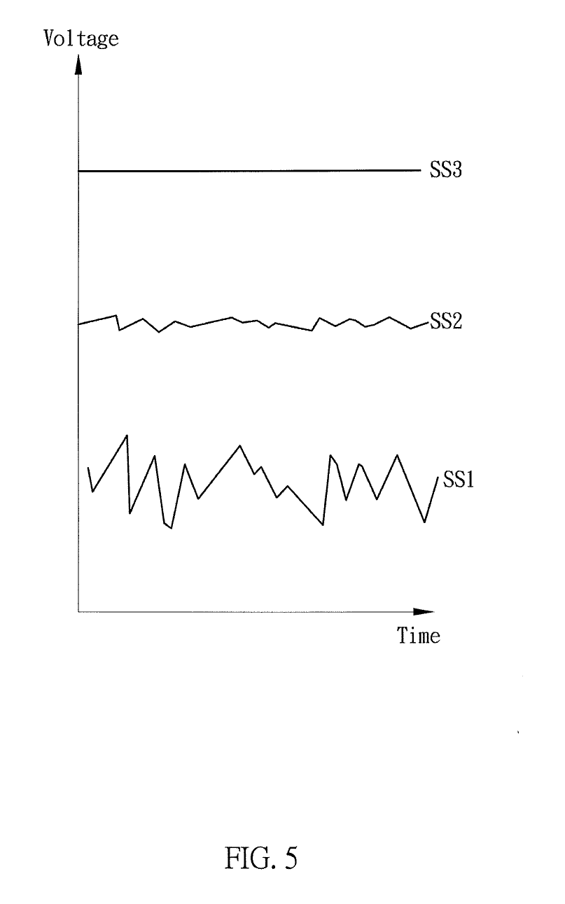

Please refer to FIG. 5. FIG. 5 illustrates diagrams of the voltages of the different sperm samples SS1˜SS3 varied with time respectively. As shown in FIG. 5, since the average voltage of the first sperm sample SS1<the average voltage of the second sperm sample SS2<the average voltage of the third sperm sample SS3, it can be found that the quantity of the first sperm sample SS1>the quantity voltage of the second sperm sample SS2>the quantity voltage of the third sperm sample SS3.

As shown in FIG. 5, since the upper and lower volatility of the voltages of the first sperm sample SS1>the upper and lower volatility of the voltages of the second sperm sample SS2>the upper and lower volatility of the voltages of the third sperm sample SS3, it can be found that the percentage of sperms having a moving speed larger than 25 um/sec in the first sperm sample SS1>the percentage of sperms having a moving speed larger than 25 um/sec in the second sperm sample SS2>the percentage of sperms having a moving speed larger than 25 um/sec in the third sperm sample SS3.

As mentioned above, in this embodiment, the first sperm sample SS1 not only has most sperms among the three sperm samples SS1˜SS3, but also has highest percentage of sperms having a moving speed larger than 25 um/sec among the three sperm samples SS1˜SS3; the third sperm sample SS3 not only has fewest sperms among the three sperm samples SS1˜SS3, but also has lowest percentage of sperms having a moving speed larger than 25 um/sec among the three sperm samples SS1˜SS3. Therefore, after the above-mentioned initial detection, the patient of the third sperm sample SS3 should go to hospital to do more detailed examinations. It helps the patient to receive early artificial insemination treatment.

Another embodiment of the invention is a method of operating a cell mobility characteristics sensing apparatus. In this embodiment, the method is used for sensing mobility characteristics of a plurality of cells in a cell sample. The cell mobility characteristics sensing apparatus includes a laser light source, a light sensor, an analyzing chip, and a display.

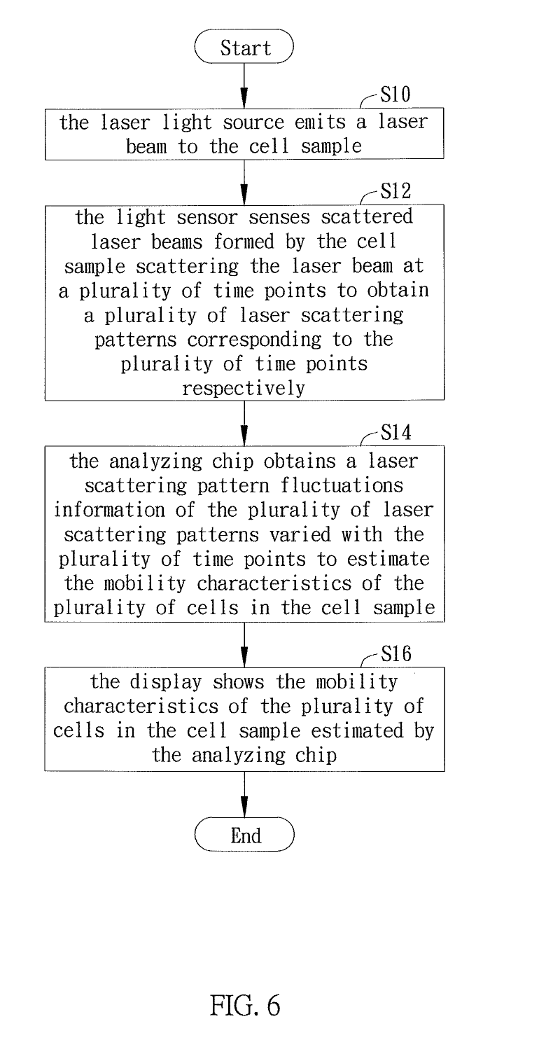

Please refer to FIG. 6. FIG. 6 illustrates a flowchart of the cell mobility characteristics sensing apparatus operating method in this embodiment. As shown in FIG. 6, in the step S10, the laser light source emits a laser beam to the cell sample. In the step S12, the light sensor senses scattered laser beams formed by the cell sample scattering the laser beam at a plurality of time points to obtain a plurality of laser scattering patterns corresponding to the plurality of time points respectively. In the step S14, the analyzing chip obtains a laser scattering pattern fluctuations information of the plurality of laser scattering patterns varied with the plurality of time points to estimate the mobility characteristics of the plurality of cells in the cell sample. In the step S16, the display shows the mobility characteristics of the plurality of cells in the cell sample estimated by the analyzing chip.

In practical applications, the mobility characteristics can be mobility, quantity, moving velocity distribution, or linearity of moving path. The light sensor can obtain the plurality of voltages corresponding to the plurality of laser scattering patterns through a photoresistor or a photodiode. The laser scattering pattern fluctuations information can include upper and lower volatility of a plurality of voltages corresponding to the plurality of laser scattering patterns varied with the plurality of time points. The more the upper and lower volatility of the plurality of voltages varied with the plurality of time points, the higher the mobility of the plurality of cells in the cell sample estimated by the analyzing chip.

Compared to the prior art, the cell mobility characteristics sensing apparatus and the operating method thereof have advantages of:

(1) simple structure and low cost;

(2) capable of calculating many cell parameters such as mobility, quantity, moving velocity distribution, and linearity of moving path and being widely applied to sense different kinds of cells;

(3) easy and fast operation for the patient to do the initial sensing of the cell mobility at home; if the sensing result is abnormal, the patient can go to hospital to do more detailed examinations.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A cell mobility characteristics sensing apparatus, for sensing mobility characteristics of a plurality of cells in a cell sample, the cell mobility characteristics sensing apparatus comprising:
a laser light source, for emitting a laser beam to the cell sample;
a light sensor, for sensing scattered laser beams formed by the cell sample scattering the laser beam at a plurality of time points to obtain a plurality of laser scattering patterns corresponding to the plurality of time points respectively;
an analyzing chip, coupled to the light sensor, for obtaining a laser scattering pattern fluctuations information of the plurality of laser scattering patterns varied with the plurality of time points to estimate the mobility characteristics of the plurality of cells in the cell sample; and
a display, coupled to the analyzing chip, for showing the mobility characteristics of the plurality of cells in the cell sample estimated by the analyzing chip.

2. The cell mobility characteristics sensing apparatus of claim 1, wherein the mobility characteristics are mobility, quantity, moving velocity distribution, or linearity of moving path.

3. The cell mobility characteristics sensing apparatus of claim 1, wherein the laser scattering pattern fluctuations information comprises upper and lower volatility of a plurality of voltages corresponding to the plurality of laser scattering patterns varied with the plurality of time points.

4. The cell mobility characteristics sensing apparatus of claim 3, wherein the more the upper and lower volatility of the plurality of voltages varied with the plurality of time points, the higher the mobility of the plurality of cells in the cell sample estimated by the analyzing chip.

5. The cell mobility characteristics sensing apparatus of claim 3, wherein the light sensor obtains the plurality of voltages corresponding to the plurality of laser scattering patterns through a photoresistor or a photodiode.

6. A method of operating a cell mobility characteristics sensing apparatus, for sensing mobility characteristics of a plurality of cells in a cell sample, the cell mobility characteristics sensing apparatus comprising a laser light source, a light sensor, an analyzing chip, and a display, the method comprising steps of:
(a) the laser light source emitting a laser beam to the cell sample;
(b) the light sensor sensing scattered laser beams formed by the cell sample scattering the laser beam at a plurality of time points to obtain a plurality of laser scattering patterns corresponding to the plurality of time points respectively;
(c) the analyzing chip obtaining a laser scattering pattern fluctuations information of the plurality of laser scattering patterns varied with the plurality of time points to estimate the mobility characteristics of the plurality of cells in the cell sample; and
(d) the display showing the mobility characteristics of the plurality of cells in the cell sample estimated by the analyzing chip.

7. The method of claim 6, wherein the mobility characteristics are mobility, quantity, moving velocity distribution, or linearity of moving path.

8. The method of claim 6, wherein the laser scattering pattern fluctuations information comprises upper and lower volatility of a plurality of voltages corresponding to the plurality of laser scattering patterns varied with the plurality of time points.

9. The method of claim 8, wherein the more the upper and lower volatility of the plurality of voltages varied with the plurality of time points, the higher the mobility of the plurality of cells in the cell sample estimated by the analyzing chip.

10. The method of claim 8, wherein the light sensor obtains the plurality of voltages corresponding to the plurality of laser scattering patterns through a photoresistor or a photodiode.
